(19) United States
(12) Patent Application Publication
(10) Pub. No.: US 2026/0181759 A1
(43) Pub. Date: Jun. 25, 2026

(54) LIGHT SWITCH UNIT FOR UV LIGHT SOURCE WITH MOTION SENSOR

(71) Applicant: CH Reynolds Electric Inc., San Jose, CA (US)

(72) Inventors: Chuck Reynolds, San Jose, CA (US); Mark Hiura, San Jose, CA (US)

(21) Appl. No.: 18/991,151

(22) Filed: Dec. 20, 2024

(51) Int. Cl. H05B 47/165; A61L 2/10; F21S 8/04; F21V 23/04; F21Y 113/00; F21Y 115/10; F24F 11/89; F24F 13/22; H05B 47/115; H05B 47/175; H05B 47/19

(52) U.S. Cl. CPC H05B 47/165; A61L 2/10; F21S 8/046; F21V 23/0435; F21V 23/0471; F24F 11/89; F24F 13/222; H05B 47/115; H05B 47/19; H05B 47/196; A61L 2103/75; F21Y 2113/30; F21Y 2115/10

(57) ABSTRACT

Embodiments of the present invention provide a reconfigurable light switch that can control multiple light sources and a fan using a combination of buttons, a motion sensor, and a timer, for improved safety and convenience. The switch can be configured to automatically activate a visible light source when motion is detected by a motion detector, or when a button exposed on the face of the light switch is pressed or otherwise activated. The light switch can also include other buttons that activate one or more fans or activate other light sources, such as a UV light source that can sanitize and disinfect the surrounding room or environment. Advantageously, the light switches of the present invention can be reconfigured using dipswitches or the like to selectively enable different modes of operation.

### FIELD

Embodiments of the present invention generally relate to the field of lighting systems. More specifically, embodiments of the present invention relate to apparatus and methods drawn to devices for controlling lighting systems.

### BACKGROUND

It is well-known that viruses, germs, bacteria, mold, and the like present a public health concern. Currently homes, businesses, and public areas are sanitized using chemicals sprayed onto surfaces and wiped away to clean the surfaces and destroy any pathogens thereon. However, this approach is time consuming, inefficient, costly, and fails to address airborne pathogens thereon that are not resting on a surface.

Recently solutions to sanitize and sterilize indoor environments incorporate the use of UV lights to destroy pathogens that may be airborne or resting on surfaces that are exposed to the UV light. However, these approaches currently rely on existing power and control systems which are not well-suited for sanitation using UV light. For example, activating UV lighting in a room occupied by people can potentially harm the occupant's skin, eyes, etc. Moreover, these lighting systems are typically controlled manually using a traditional switch which often leads to UV lights being activated during times when they are not necessarily needed or potentially dangerous to the room's occupants. A safer and more efficient light module and approach to sanitation using UV lighting are needed.

Recently innovative light systems have been introduced that include UV light sources in combination with light sources that produce visible light. In this way, the lighting system can both provide visible light as expected while at the same time offering the health and safety benefits of built-in UV sanitization. These light fixtures may also incorporate built-in exhaust fans for controlling humidity levels or circulating air. Unfortunately, existing switches and buttons used to control lighting systems are unable to fully take advantage of the benefits of these dual light source systems. Moreover, existing switches for controlling light systems cannot be easily reprogrammed or reconfigured for use with different lighting systems or in different environments.

### SUMMARY

What is needed is a switch unit for a lighting system capable of controlling both a UV light source and a visible light source, and optionally a fan, while also incorporating motion detection and timer-based technology for improved safety and efficiency. Accordingly, embodiments of the present invention provide a reconfigurable light switch that can control multiple light sources and a fan using a combination of buttons or switches, a motion sensor, and a timer, for improved safety and convenience.

According to one disclosed embodiment, a switch control unit is disclosed, including a first switch operable to control a first light source, a button operable to control a second light source configured to produce ultraviolet (UV) light, a motion detector, and control circuitry operable to selectively activate the first light source when the first switch is toggled, and automatically deactivate the first light source and activate the second light source when the button is pressed after the motion detector fails to detect motion for a predetermined countdown duration.

According to a different embodiment, a lighting assembly is disclosed, including a control unit, a first light source, a second light source configured to produce ultraviolet (UV) light, and control circuitry for selectively powering the first light source and the second light source, wherein the control circuitry is operable to automatically deactivate the first light source and activate the second light source according to a signal received from the control unit.

According to another embodiment, a switch control unit is disclosed, including a first switch operable to control a first light source, a second switch operable to control an exhaust fan, a button operable to control a second light source configured to produce ultraviolet (UV) light, a motion detector, a humidity sensor, and control circuitry operable to: selectively activate the first light source when the first switch is toggled, automatically control the exhaust fan according to a reading of the humidity sensor, and automatically deactivate the first light source and activate the second light source when the button is pressed after the motion detector fails to detect motion for a predetermined countdown duration.

### BRIEF DESCRIPTION OF THE DRAWINGS:

FIG. 1 depicts an exemplary light switch unit for controlling a dual-purpose lighting system and a fan, which can be an exhaust fan, according to embodiments of the present invention.

FIG. 2 depicts an exemplary light switch unit for controlling a dual-purpose lighting system including an ultraviolet light source and two separate visible light sources according to embodiments of the present invention.

FIG. 3 depicts components of an exemplary light switch unit for controlling a dual-purpose lighting system including an ultraviolet light source, one or more visible light sources, and optionally a fan using control circuitry according to embodiments of the present invention.

FIG. 4 depicts an exemplary light switch unit for controlling a dual-purpose lighting system, an ultraviolet light source, two visible light sources, and optionally a fan using a CPU and memory according to embodiments of the present invention.

FIG. 5 depicts an exemplary dipswitch unit including 4 dipswitches for configuring various functions of a light switch unit for controlling a multi-use light system according to embodiments of the present invention.

FIG. 6 depicts an exemplary dual-purpose lighting system including a UV light source controlled by a light switch unit according to embodiments of the present invention.

FIG. 7 depicts an exemplary dual-purpose lighting system including a UV light source and a fan each controlled by a light switch unit according to embodiments of the present invention.

FIG. 8 is a block timing diagram depicting exemplary approaches to motion-based UV light activation according to embodiments of the present invention.

### DETAILED DESCRIPTION

This application is related to and fully incorporates the disclosure of U.S. Pat. No. 12,011,511 filed on Apr. 25, 2023, previously U.S. patent application Ser. No. 18/139,317, in its entirety as if fully set forth below.

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart of the figure herein, and in a sequence other than that depicted and described herein.

### Light Switch Unit For Lighting Apparatus Including Ultraviolet Light Source

Embodiments of the present invention provide a reconfigurable light switch unit that can control multiple light sources and a fan using a combination of switches, buttons, a motion sensor, and a timer, for improved safety and convenience. The switch unit can be configured to automatically activate a visible light source when motion is detected by a motion detector, or when a button exposed on the face of the light switch is pressed or otherwise activated (e.g., touched, flipped, wound, etc.). The light switch unit can also include other switches/buttons that activate one or more fans (e.g., an overhead fan or an exhaust fan) or other light sources, such as a UV light source that can sanitize and disinfect the surrounding room or environment. Advantageously, the light switch units of the present invention can be reconfigured using dipswitches or the like to selectively enable different modes of operation. According to some embodiments, the light switch is configured to operate in a commercial or office environment. Other embodiments are well-suited for use in a bathroom or similar environment where humidity control functionality using a fan is desired. The light switch unit can also include a CPU, memory, and a wireless transceiver, according to some embodiments.

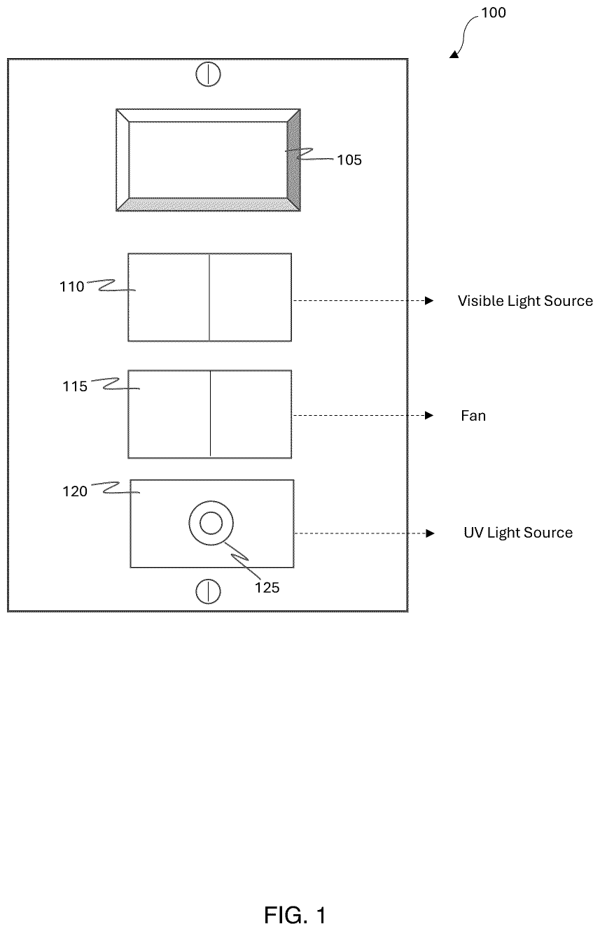

FIG. 1 depicts an exemplary light switch unit 100 (“control unit”) for controlling a dual-purpose lighting system and a fan 115, which can be an exhaust fan, maintenance fan, or similar device capable of blowing air into or out of a room, or to circulate air, according to embodiments of the present invention. The evacuation fan 115 can be built into the lighting system or included as a separate unit. The fan can operate to mitigate humidity levels inside the room where the light system is installed. According to some embodiments, button 120 activates the fan until a predetermined humidity level is achieved (e.g., 50% humidity) or based on a timer period to shut off. According to some embodiments, fan 115 includes a built-in visible light source that activates when the fan 115 is actively blowing air. Fan 115 can further be controlled based on motion detected by motion detector 105 (or by a separate motion detector), and can be controlled according to a predetermined schedule or by signals transmitted by a wireless device.

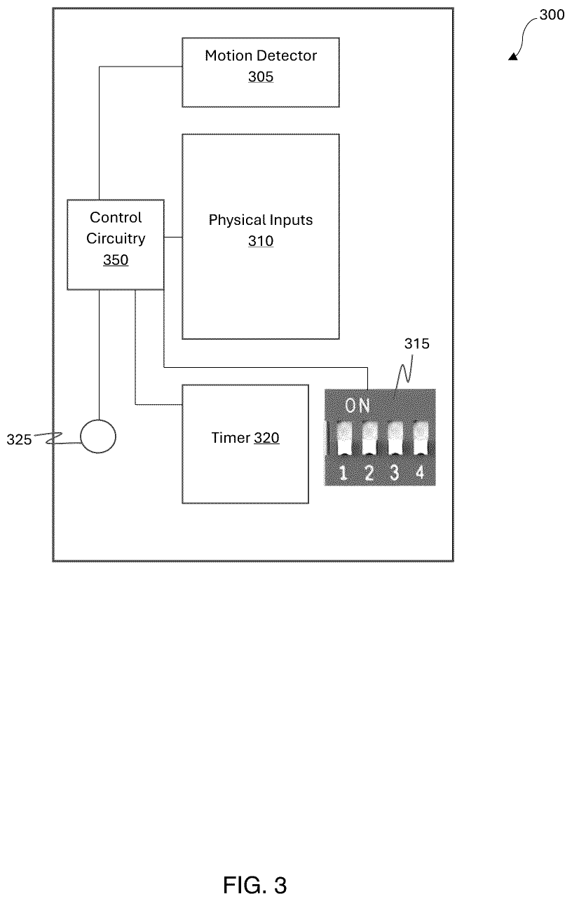

The dual-purpose lighting system connected to light switch unit 100 typically includes a set of visible light sources (e.g., LED, halogen, florescent, etc.) that produce visible light, and a set of UV light sources for sanitation, which are typically LEDs. As depicted in FIG. 1, light switch unit 100 includes a motion detector 105, buttons or switches 110, 115 120, and a status light (e.g., an LED) 125 integrated into UV button 120. It should be noted that the functions of light switch unit 100 can be adjusted to different modes or configurations by adjusting dipswitches disposed within light switch unit 100, as depicted in the examples of FIGS. 3-5. Moreover, while status light 125 is integrated into UV button 120 having a semi-transparent body in the embodiment of FIG. 1, status light 125 can be integrated into another portion of light switch unit 100, and/or can be included as a separate component remote from light switch 100, according to embodiments.

According to one exemplary configuration, light switch unit 100 operates to activate the visible light source when button 110 is pressed. Button 110 can include a push button or toggle switch, for example, that activates the visible light source until the visible light source is deactivated by pressing or toggling button/switch 110 a second time. According to some embodiments, the visible light source is automatically turned off is automatically turned off when no motion is detected for a prolonged duration (e.g., 2-8 hours).

Pressing button/switch 115 can activate the exhaust fan, and pressing button/switch 115 again deactivates the exhaust fan. The fan can also be automatically deactivated based on humidity levels in the room or based on a timer expiring, according to embodiments. Button 120 is a momentary contact button used to activate the UV light source. The light switch unit 100 typically controls the activation of the visible light source and the fan by opening or closing one or more power relays coupled (e.g., normally open and normally closed relays) to the visible light source and the fan (e.g., a normally open or normally closed relay), although other means of activating the light sources and the fan can be used (e.g., wireless activation signals). The power source can be a 120-277V power source, for example.

Importantly, the UV light source does not activate immediately when button 120 is pressed. Instead, after a brief delay (e.g., 10 seconds), a timer is activated, and the UV light source does not activate until no motion is detected by motion detector 105 for a predetermined countdown duration. Once the timer expires with no motion detected, the UV light source is automatically activated and the visible light is shut off. In this way, the UV light source should only activate when no one is present inside the room and it is safe to sanitize the room with UV light. The predetermined duration can be equal to 10 second, 30 seconds, or 60 seconds, for example. Once activated, the UV light source remains active for a second predetermined duration, typically 15-20 minutes, or until the desired level of sanitation is achieved. With the UV light on, any detected motion will turn off the UV light source and optionally turn on the visible light source (and may cause status light 125 to flash. According to some embodiments, the UV lights remain active indefinitely until motion is detected or a specific control signal is received. According to some embodiments, the UV lights are only activated when the visible light source is not turned on. According to some embodiments, the UV lights turn on automatically after a countdown duration any time the visible light source is not turned on and no motion is detected for a predetermined countdown duration.

Status light 125 visually indicates the status of the UV light source. For example, status light 125 can blink or flash during the countdown period after UV button 220 is activated, and status light 125 can stay consistently lit while the UV light source is active. In this way the user is able to easily recognize that the UV light source will soon be activated and make sure that the room remains vacant during the sanitation duration. The status light 125 can also blink any time motion is detected by motion detector 105. If motion is detected after the countdown period, then the UV light source is deactivated.

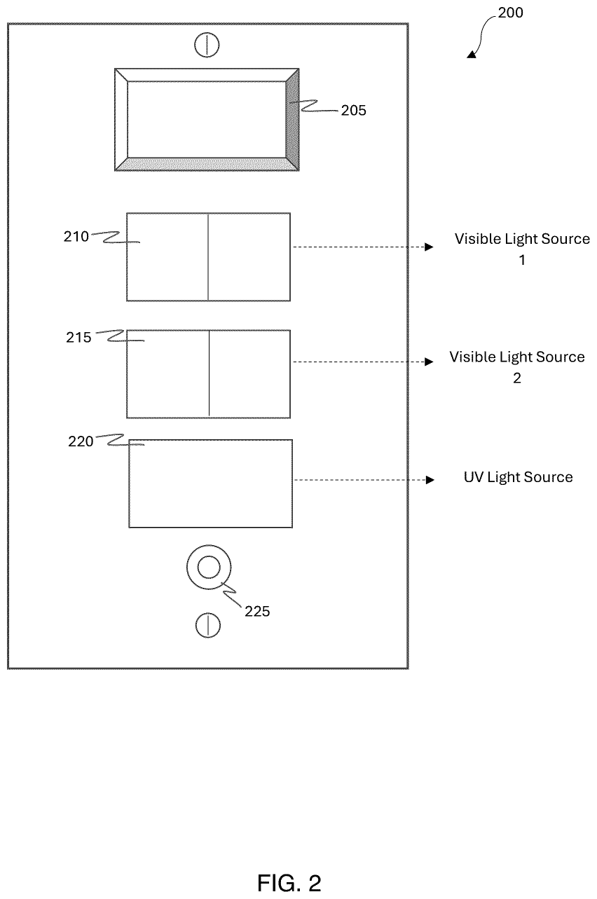

FIG. 2 depicts an exemplary light switch unit 200 for controlling a dual-purpose lighting system including an ultraviolet light source and two separate visible light sources according to embodiments of the present invention. Similar to the embodiment depicted in FIG. 1, light switch unit 200 includes a motion detector 205, buttons or switches 210, 215, 220, and a status light (e.g., LED) 225. However, in the example of FIG. 2, button or switch 215 is configured to control a second visible light source instead of a fan. This configuration can be achieved by toggling a dipswitch of light switch unit 200, according to some embodiments. Buttons 210 and 215 can be configured to control different light sources of a split overhead commercial lighting system, for example, and can be activated automatically when motion is detected, according to embodiments.

Button 220 is a momentary contact button and controls a UV light source that is activated after a brief delay and a predetermined countdown if no motion is detected by motion detector 205 during the duration of the countdown. As mentioned above, the functionality of exemplary light switch unit 200 can be configured by adjusting one or more dipswitches disposed within light switch unit 200. According to other embodiments, light switch unit 200 includes one or more radios or wireless transceivers capable of wireless communication, and the functionality of exemplary light switch unit 200 can be adjusted using a smartphone or similar wireless communication device over Bluetooth, Wi-Fi, etc.

FIG. 3 depicts an exemplary light switch unit 300 for controlling a dual-purpose lighting system including an ultraviolet light source, one or more visible light sources, and optionally a fan according to embodiments of the present invention. Exemplary light switch unit 300 includes a motion detector 305, multiple physical inputs (e.g., buttons, switches, levers, etc.) 310 for controlling the light sources or the fan using control circuitry 350, dipswitches 315 for configuring the functionality of light switch unit 300, timer 320, and status light 325. Status light 325 can be coupled to timer 320 and motion detector 305 to indicate the status of those components (e.g., motion detected, timer countdown, UV light source activated, etc.). Control circuitry 350 disposed within light switch unit 300 allows the motion detector 305, physical inputs 310, and timer 320 to communicate electronically to control the light sources and/or fans according to the positions of dipswitches 315. Control circuitry 350 could include a processor or state machine or any control electronics. According to some embodiments, status light 325 is integrated into one of the physical inputs 310 having a semi-transparent housing.

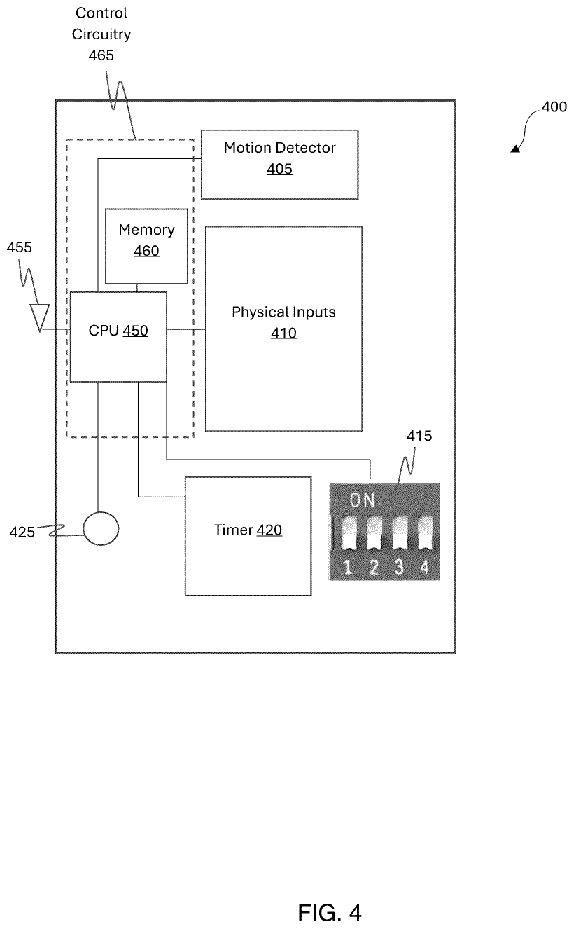

FIG. 4 depicts an exemplary light switch unit 400 for controlling a dual-purpose lighting system, an ultraviolet light source, one or more visible light sources, and optionally a fan using control circuitry 465 including a processor 450 according to embodiments of the present invention. Exemplary light switch unit 400 includes a motion detector 405, multiple physical inputs (e.g., buttons, switches, levers, etc.) 410 for controlling light sources and/or fans, dipswitches 415 for configuring the functionality of light switch unit 400 (optional in this embodiment), timer 420, and status light 425 using logic executed by processor 450. Memory 460 is coupled to CPU 450 and can store configuration data (e.g., switch/button configuration, countdown duration, sanitation duration, wireless security information, etc.). Status light 425 can be coupled to timer 420 and motion detector 405 to indicate the status of those components (e.g., motion detected, timer countdown, UV light source activated, etc.). Light switch unit 400 can further include one or more wireless radios 455 coupled to processor 450 to enable wireless communications with other devices (e.g., smartphones) over Wi-Fi-, Bluetooth, Zigbee, Z-wave, and the like. Using wireless radio 455, light switch unit 400 can receive control commands to activate or deactivate the visible light source, the UV light source, a fan, etc. Configuration data can also be received wirelessly, and can override the configuration of dipswitches 415. According to some embodiments, processor 450 can receive scheduling information over the wireless radio 455 to precisely control when various light sources or fans are active, using an on/off schedule, a weekly schedule, etc.

According to various embodiments, processor 450 can be a CPU, FPGA, a custom integrated circuit, a state machine, or the like, operable to control the functionality of light switch unit 400 and to send and receive data using a wireless radio included in light switch unit 400. Processor 450 can receive timer duration information, sanitation duration information, and other configuration information for controlling the functionality of physical inputs 410 that control different light sources, fans, etc. and the information can be stored in memory 460.

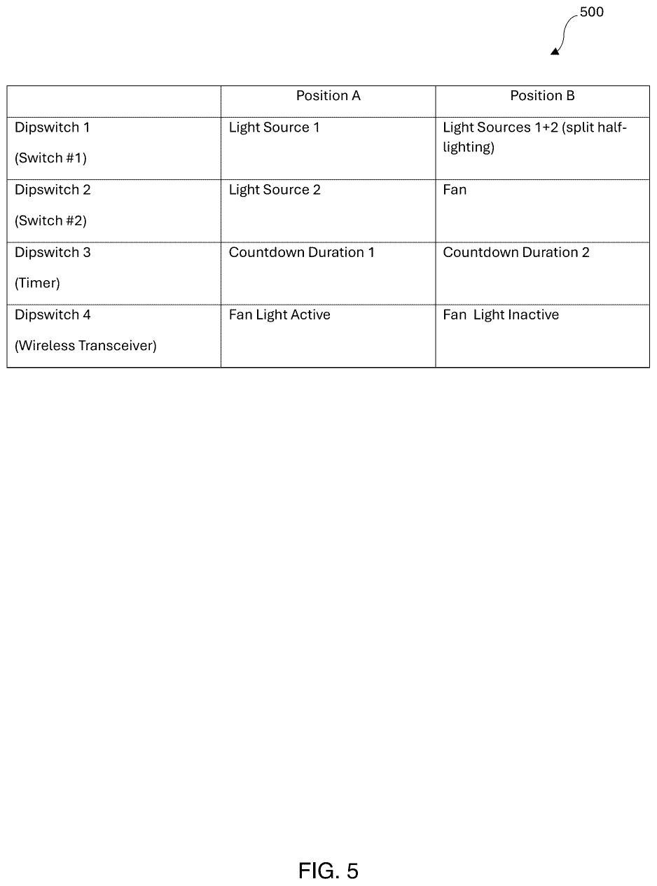

FIG. 5 depicts an exemplary dipswitch unit 500 including 4 dipswitches for controlling various functions of a light switch unit for controlling a multi-use light system according to embodiments of the present invention. Dipswitch unit 500 is typically integrated into a light switch unit described herein according to embodiments of the present invention. Each dipswitch can be toggled in one of two positions, as desired, and the corresponding light switch unit is automatically configured according to the position of each dipswitch.

In the example of FIG. 5, dipswitch 1 corresponds to the first button or switch of light switch unit (e.g., button 110 or 210) and can be toggled to control the light source 1 or both light source 1 and light source 2. According to some embodiments, toggling dipswitch 1 enables/disables half-lighting, where half of the power is directed towards light source 1 and the other half of the power is directed towards light source 2.

Dipswitch 2 corresponds to the second button or switch of the light switch unit (e.g., button 115 or 215) and can be toggled to control light source 2 or a fan (e.g., an exhaust fan or maintenance fan).

Dipswitch 3 corresponds to the timer of the light switch unit and can be toggled to adjust the countdown duration before the UV light source is activated when no motion is detected during the countdown duration. For example, one position of dipswitch 3 can correspond to a regular countdown duration, and the other position can correspond to a shorter or longer countdown duration.

Dipswitch 4 controls an optional light source built-into a fan (optional) controlled by the third button of the light switch unit (e.g., button 120 or 220). According to other embodiments, dipswitch 4 can be assigned to control other functionality, such as activating or deactivating a wireless radio of light switch unit 550, adjusting the sanitation duration (when the UV light source is active), or setting the desired humidity level when a fan is coupled to the lighting system.

While the various light sources, fans, and light switch units are typically hard wired together, according to some embodiments, the light switch unit communicates with the light sources and/or fans wirelessly, using Wi-Fi-, Bluetooth, Zigbee, Z-wave, etc. According to some embodiments, the light sources, fans, and light switch unit share a common power source. According to other embodiments, the light sources, fans, and light switch unit may be powered by separate power sources.

### Indoor Lighting Apparatus Including Ultraviolet Light Source

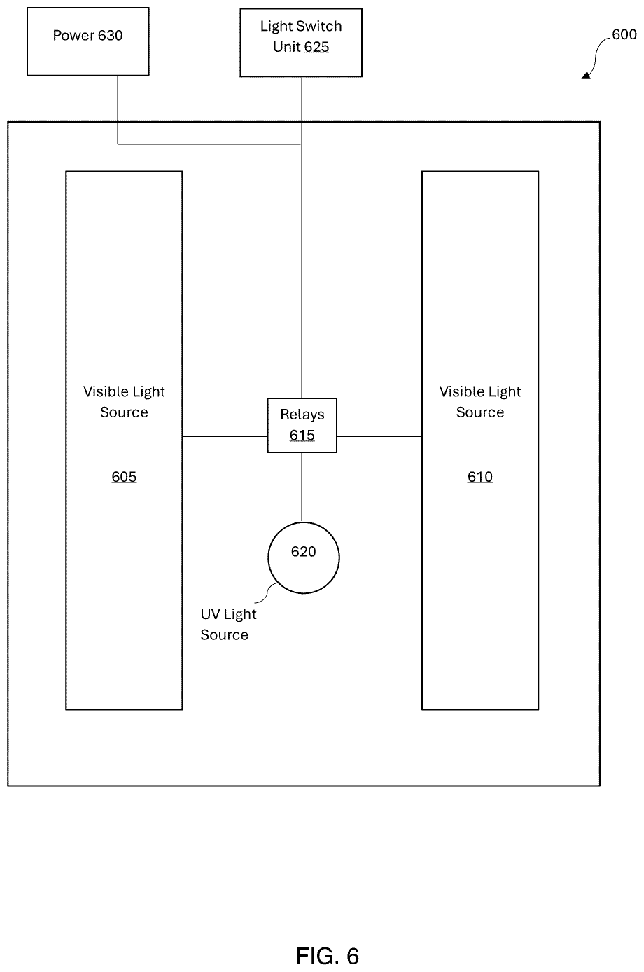

FIG. 6 depicts an exemplary lighting assembly or fixture 600 with two visible light sources 605, 610 and a controllable relays 615 operated by a light switch unit 625 according to embodiments of the present invention. The relays 615 can be controlled (opened/closed) by selectively energizing an electromagnet in the relay using a power source (e.g., power source 630) in one embodiment. Light switch unit 625 includes one or more physical inputs, such as buttons or switches, that control the light sources as described above with respect to FIGS. 1, 2. The functions of light switch unit 625 can be configured by one or more dipswitches as described above with respect to FIG. 5.

Lighting assembly 600 produces visible light using visible light source 605 and/or visible light source 610. Two visible light emitters are shown but this is exemplary only, as the light fixture 600 can include fewer or more visible light emitters. Visible light source 605 and visible light source 610 are coupled to light couplings that both secure the lights and also conduct power to the lights; visible light source 605 and visible light source 610 can include fluorescent overhead lights, LED lighting arrays, or any suitable light source configured to provide visible light. Light source 620 on the other hand is a light source configured to produce UV light, such as an LED, and is secured by light couplings which also conduct power to UV light source 620 and can be used to secure UV light source 620. Any suitable means for producing UV light can be used.

The UV light as is known cleans and/or disinfects the environment generally nearby the lighting fixture 600, e.g., about 20 feet from the fixture, over a period of exposure equal to about 30-45 minutes. The duration of the UV light produced by UV light source 620 can be controlled by a timer and/or a motion detector of light switch unit 625. For instance, if no motion is detected over a period of time by the motion detector, then UV light source 620 can be turned on. The timer can be set for a predetermined duration (e.g., 5-15 minutes) and the duration can be adjusted by dipswitches of light switch unit 625, or by commands received wirelessly from a wireless device (e.g., smartphone). During the “on” duration period of the UV light, any motion detected by the motion detector shuts off the UV light and turns off the UV “on” period.

The timer signal can be used to provide the desired amount of UV light and another timer signal can also be used as an inhibitor signal to prevent the UV light from being active when people are expected to enter the environment (e.g., an office, store, home, or other similar indoor environment) during work hours, for example. Saying this another way, a timer signal can be used as an override to only allow the UV “on” duration during those hours when people are not expected to be in the room, e.g., after regular work hours of an office or during expected sleep periods of a home. The lighting schedule can be defined by an application executed by a wireless device that transmits configuration data to light switch unit 625 e.g., over a wireless channel.

Lighting assembly 600 includes relays 615 for selectively powering visible light sources 605, 610 to produce visible light, as well as UV light source 620 to provide UV light. The relays 615 are coupled to a power source and a motion sensor that can selectively open/close the relay when motion is detected to produce visible light (and turn off Light source 3) for the detected person/motion. Relays 615 can include any number of normally open or normally closed relays to control activation of the various light sources using respective switches or buttons. For example, relays 615 can be configured in the open position to provide power to visible light sources 605, 610. It is appreciated that UV light source 620 is not powered when relays 615 are configured in this way as UV light is not advised when people are present. Therefore, in the condition when motion is detected, the lighting assembly 600 does not produce UV light and the environment is lit with visible light and is safe for occupants.

In one exemplary embodiment, when the motion sensor does not detect motion for a prescribed period of time, the room is assumed to be empty, and the motion sensor can cause s respective relay of relays 615 to close. According to other embodiments, relays 615 include a normally closed relay that remains closed until toggled by the motion sensor so that UV light source 620 can be powered to produce UV light. UV light source 620 can be further controlled by a timer signal so that the UV light is only produced when the timer is active (not yet expired) and defines a UV “on” duration. Again, the UV “on” duration does not trigger unless motion has not been detected for the prescribed period. The UV “on” duration will terminate upon the motion detector detecting motion.

It is further appreciated that the UV “on” duration can be further activated as described above, by another (second) timer signal which defines periods when people are not expected to be present, e.g., during non-working hours or typical sleep times. In this embodiment, the UV “on” duration will not start unless 1) motion has not been detected over the prescribed period AND 2) the second timer indicates a safe period, e.g., people are not expected to be present. When 1) and 2) are satisfied, in this embodiment, then the UV on period starts and will run for the timer duration unless motion is detected again. It is appreciated that the source of the second timer can be integrated within the fixture 600 or it can be generated by a timer or processor of light switch unit 625.

According to some embodiments, lighting assembly 600 includes additional relays/circuitry so that visible light source 605 and visible light source 610 can be powered or activated separately. For example, only one of visible light source 605 and visible light source 610 can be powered to provide half of the visible light that can be produced by lighting assembly 600, and both of visible light source 605 and visible light source 610 can be powered at the same time to provide the full amount of visible light. In other embodiments visible light source 605 and visible light source 610 are also dimmable.

According to some embodiments, visible light source 605 and visible light source 610 are a single light source.

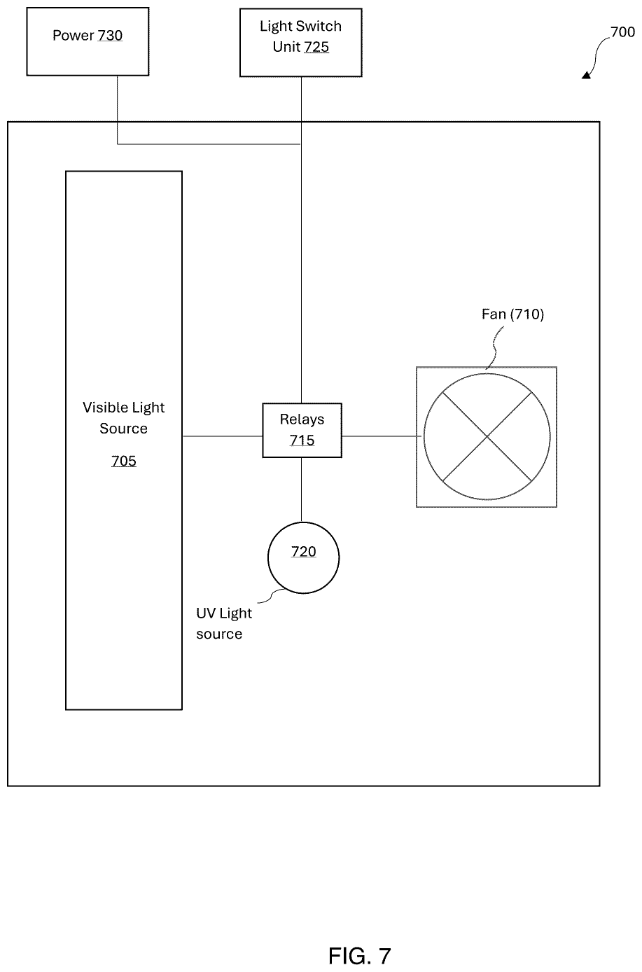

FIG. 7 depicts an exemplary lighting assembly or fixture 700 with a visible light source 705, an exhaust fan 710, and a controllable relay 715 operated by a light switch unit 725 according to embodiments of the present invention. The relay 715 can be controlled (opened/closed) by selectively energizing an electromagnet in the relay using a power source (e.g., power source 730) in one embodiment.

Lighting assembly 700 produces visible light using visible light source 705 and circulates air using exhaust fan 710. When fan 710 is active, air is forcibly blown into or out of the environment. According to some embodiments, lighting assembly or fixture 700 include a humidity sensor, which can be disposed within light switch unit 725 or installed separately, and fan 710 remains active until a prescribed humidity level is reached or until a timer period runs out. The desired humidity level is determined by light switch unit 725 and can be adjusted using a dipswitch or wireless electronic device. Typically fan 710 is activated manually by pressing a button or switch of light switch unit 725, although fan 710 can be activated automatically by light switch unit 725 according to a schedule or based on humidity levels of the environment.

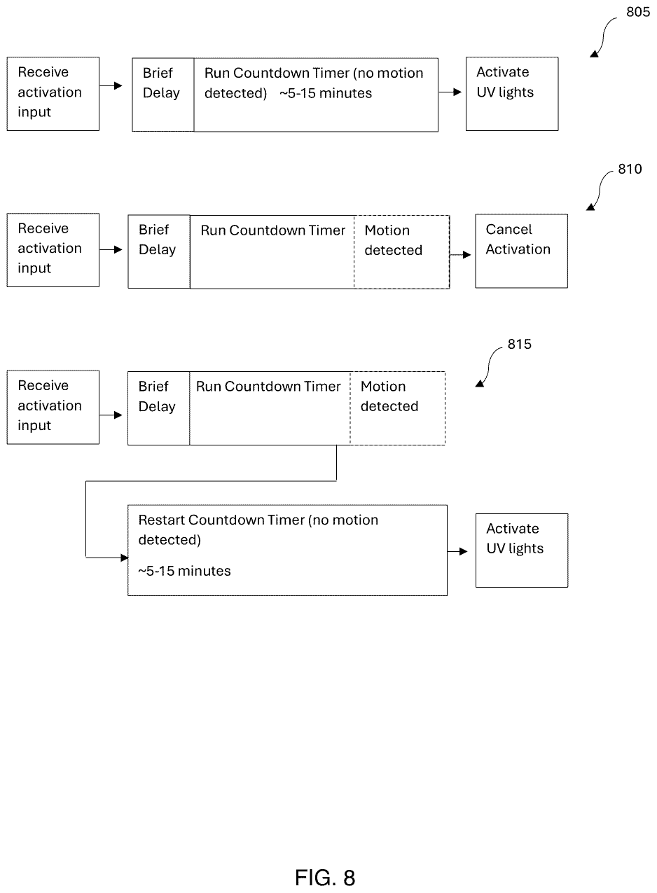

FIG. 8 is a block timing diagram depicting exemplary steps for motion-based UV light activation according to embodiments of the present invention. In process 805, receiving activation input starts a countdown timer, which is typically configured for 10-30 minutes, and the countdown timer begins after a short delay to allow occupants to leave the room (e.g., 10 seconds). Lower power UV lights (e.g., 6 watts) may use a longer duration (e.g., 1-8 hours). When no motion has been detected and the countdown timer expires, the UV lights are activated. In process 810, receiving activation input starts a countdown timer after a brief delay, and detecting motion before the countdown expires causes the activation of the UV lights to be canceled. In process 815, receiving activation input starts a countdown timer, and after a brief delay, detecting motion before the countdown expires causes the countdown timer to be restarted. This process can be repeated any number of times until no motion is detected during the countdown period (e.g., 3-5 minutes), in which case the UV lights are activated.

According to some embodiments, the light sources, sensors, fans, etc., are Power-over-Ethernet (PoE) devices that are powered by electrical power over Ethernet using the same cable or cables that provides the network traffic.

Embodiments of the present invention are thus described. While the present invention has been described in particular embodiments, it should be appreciated that the present invention should not be construed as limited by such embodiments, but rather construed according to the following claims.

What is claimed is:

1. A switch control unit comprising:
a first switch operable to control a first light source;
a button operable to control a second light source configured to produce ultraviolet (UV) light;
a motion detector; and
control circuitry operable to:
selectively activate the first light source when the first switch is toggled; and
automatically deactivate the first light source and activate the second light source when the button is pressed after the motion detector fails to detect motion for a predetermined countdown duration.

2. The switch control unit of claim 1, wherein the first and second light sources are disposed within a housing, and wherein the housing is operable to be mounted as an overhead lighting assembly for installation in a ceiling of an indoor environment.

3. The switch control unit of claim 1, wherein the first light source produces visible light and comprises at least one of: a plurality of fluorescent lights and a plurality of visible light LED lights, and wherein the control circuitry is operable to turn off the first light source if no motion is detected by the motion detector after a prescribed period.

4. The switch control unit of claim 1, wherein the second light source comprises a UV LED emitter, and wherein the button comprises a momentary contact button.

5. The switch control unit of claim 1, further comprising:
a timer, wherein the predetermined countdown duration is controlled by the timer; and
an LED indicator operable to provide an indication that the timer is actively counting down the predetermined countdown duration before the second light source is activated.

6. The switch control unit of claim 5, further comprising a wireless transceiver, wherein the wireless transceiver is operable to receive configuration data from a wireless device to configure the timer.

7. The switch control unit of claim 5, wherein the wireless transceiver is further operable to receive a deactivation signal that deactivates the second light source.

8. The switch control unit of claim 5, further comprising:
a second switch;
a first dipswitch operable to configure the first switch to control at least one of:
the first light source; and a third light source;
a second dipswitch operable to configure the second switch to control at least one of: the first light source; and a fan.

9. The switch control unit of claim 8, further comprising a third dipswitch operable to adjust a length of the predetermined countdown duration.

10. The switch control unit of claim 8, further comprising:
a wireless transceiver; and
a third dipswitch operable to selectively activate and deactivate the wireless transceiver.

11. A lighting assembly comprising:
a control unit;
a first light source;
a second light source configured to produce ultraviolet (UV) light; and
control circuitry for selectively powering the first light source and the second light source, wherein the control circuitry is operable to automatically deactivate the first light source and activate the second light source according to a signal received from the control unit.

12. The lighting assembly of claim 11, wherein the control circuitry further comprises: a CPU; and a wireless transceiver, wherein the CPU is operable to access signals from the wireless transceiver to configure the control unit.

13. The lighting assembly of claim 12, further comprising a timer, wherein the signals comprise commands to adjust a length of the timer, wherein the timer is operable to delay activation of the second light source.

14. The lighting assembly of claim 12, wherein the signals are operable to activate or deactivate at least one of: the first light source; and the second light source.

15. The lighting assembly of claim 12, wherein the signals comprise a schedule of times when the second light source will be activated automatically by the control circuitry.

16. The lighting assembly of claim 11, further comprising:
a timer; and
a dipswitch, wherein the dipswitch is operable to adjust a length of the timer, wherein the timer is operable to delay activation of the second light source.

17. The lighting assembly of claim 11, wherein the control unit comprises a motion sensor, and wherein the control circuitry is operable to activate the second light source when the motion sensor fails to detect motion for a predetermined countdown duration.

18. The lighting assembly of claim 17, wherein the control unit comprises a dipswitch operable to adjust a length of the predetermined countdown duration.

19. The lighting assembly of claim 17, wherein the control unit comprises:
a dipswitch;
a first switch operable to receive input to control the first light source; and
a second switch operable to receive input to control at least one of: a fan; and a third light source, according to a configuration of the dipswitch.

20. The lighting assembly of claim 11, further comprising:
a humidity sensor; and
a fan, wherein the control circuitry automatically controls the fan according to a reading of the humidity sensor.

21. A switch control unit comprising:
a first switch operable to control a first light source;
a second switch operable to control an exhaust fan;
a motion detector;
a humidity sensor; and
control circuitry operable to:
selectively activate the first light source when the first switch is toggled;
automatically control the exhaust fan according to a reading of the humidity sensor; and
automatically deactivate the first light source and activate a second light source after the motion detector fails to detect motion for a predetermined countdown duration.

22. The switch control unit of claim 21, wherein the first and second light sources and the exhaust fan are disposed within a housing, and wherein the housing is operable to be mounted as an overhead lighting assembly for installation in a ceiling of an indoor environment.

23. The switch control unit of claim 21, wherein the first light source produces visible light and comprises at least one of: a plurality of fluorescent lights and a plurality of visible light LED lights, and wherein the control circuitry is operable to turn off the first light source if no motion is detected by the motion detector after a prescribed period.

24. The switch control unit of claim 21, wherein the second light source comprises a UV LED emitter, and further comprising a momentary contact button that controls the second light source when no motion is detected by the motion sensor for the predetermined countdown duration after the momentary contact button is pressed.

25. The switch control unit of claim 21, wherein the exhaust fan comprises a fan light that is automatically activated when the exhaust fan is active.
